(19) United States
(12) Patent Application Publication
(10) Pub. No.: US 2005/0225326 A1
(43) Pub. Date: Oct. 13, 2005

(54) Signal receiving method and device for a magnetic resonance imaging system

(76) Inventors: Zeng He He, ShenZhen (CN); Xiao Fei Liu, ShenZhen (CN); Jian Min Wang, ShenZhen (CN); Ting Qiang Xue, ShenZhen (CN)

Correspondence Address: SCHIFF HARDIN, LLP;PATENT DEPARTMENT, 6600 SEARS TOWER, CHICAGO, IL, 60606-6473, US

(21) Appl. No.: 11/095,230

(22) Filed: Mar. 31, 2005

(30) Foreign Application Priority Data
Mar. 31, 2004 (CN) 200410029671.7

(51) Int. Cl. G01V 3/00; A61B 5/05

(52) U.S. Cl. 324/318; 324/309; 600/416

(57) ABSTRACT

In a signal receiving method and device for a magnetic resonance imaging system, a first loop receiver coil has a saddle receiver coil overlapped thereon, and two individual loop receiver coils or two counter rotating loop receiver coils connected end-to-end are equally spaced from the first loop receiver coil along the axial direction of the first loop receiver coil, forming a dual-loop receiver coil unit. Thus, the magnetic resonance signal generated by human tissue induces current on the dual-loop receiver coil unit following excitation of the signal. When the device is operated, the signal current induced in the saddle receiver coil, the first loop receiver coil and the dual-loop receiver coil unit respectively are added, thus increasing the output signal-to-noise ratio, improving the edge imaging effect, increasing the coverage of the coil signal and improving the signal homogeneity in the imaging area.

### BACKGROUND OF THE INVENTION

1. Field of the Invention

The present invention relates to an antenna device for receiving signals in a magnetic resonance imaging system and a signal receiving method. In particular, the present invention relates to a receiving antenna device in a magnetic resonance imaging system and a method for adjusting the operation mode of the receiver coils to enhance the signal-to-noise ratio of the output signals from the device and improve the signal homogeneity in the imaging area and the edge property.

The present invention is adapted to detect the magnetic resonance image signal induced by human limbs, and animal limbs similar to human limbs or trunk.

2. Description of the Prior Art

In conventional receiving antenna devices for a magnetic resonance imaging system that receive signals such as a signal from a human knee joint, the basic structural unit of the antenna for receiving signals is usually a combined receiver coil formed by a first loop receiver coil and a saddle receiver coil, wherein the first loop receiver coil mainly assures adequate signal intensity in the middle of the imaging area, while the saddle receiver coil helps to realize a field of view (FOV) or receiving region of signals with certain dimensions. In comparison to using only one first loop receiver coil, this type of coil set structure can theoretically expand the imaging area and improve the imaging quality. However, under current technical constraints, due to the weak signal receiving ability of the saddle coil the actual effect of expanding the imaging area is quite limited, and there is no recognizable improvement in imaging quality. Moreover, the imaging quality for the edge portions on both sides of the imaging area will deteriorate significantly.

### SUMMARY OF THE INVENTION

An object of the present invention is to provide an improved signal receiving method and an improved signal receiving device for a magnetic resonance imaging system to expand the effective field of viewing of signals, enhance the signal-to-noise ratio, improve the signal homogeneity, and the imaging effect for the edge portions.

To overcome the aforementioned deficits of conventional signal receiving antenna devices for a magnetic resonance imaging system, the present invention adds a dual-loop receiver coil unit on the basis of the original first loop receiver coil and saddle receiver coil. The dual-loop receiver coil unit can be formed by two counter-rotating loops (CR-LOOP), wherein the two counter-rotating loops are connected end-to-end in series, and two independent loops.

Thus, in accordance with the present invention, a signal receiving method for a magnetic resonance imaging system, wherein the direction of the static magnetic field of the magnetic resonance imaging system is along the Z axis direction, includes:
(1) providing a first loop receiver coil, surrounding the middle of the imaging (detecting) area, for detecting the signal intensity in the middle of the imaging area, wherein the axial direction of the first loop receiver coil is along the Y axis direction;
(2) overlapping a saddle receiver coil on the outside of the first loop receiver coil in the radial direction, for guaranteeing a certain signal intensity of the entire imaging area, wherein the direction of the two opposite curved surfaces of the saddle receiver coil is along the X axis direction;
(3) providing a dual-loop receiver coil unit, formed by a second loop receiver coil and a third loop receiver coil, wherein the axial direction of the two loop receiver coils is along the Y axis direction and they are respectively disposed on opposite sides along the Y axis direction relative to the first loop receiver coil, and are substantially equally spaced from the first loop receiver coil, for causing the magnetic resonance signal generated by the detected living organism to induce current in the two loop receiver coils of the dual-loop receiver coil unit following excitation of the signal;
(4) supplying the detected signals of the first loop receiver coil, the saddle receiver coil, and the second and third loop receiver coil units to at least one radiofrequency receiving channel and combining al of the detected signals; and
(5) adjusting the positions of the second loop receiver coil and the third loop receiver coil relative to the first loop receiver coil for optimizing the reception of the magnetic resonance signal of the detected living organism, dependent on one or more of the following factors:
1) the signal homogeneity of the imaging area of the induced magnetic resonance signals in the second loop receiver coil and the third loop receiver coil,
2) the expansion of the imaging area,
3) the improvement of the imaging of the edge portions of the imaging area,
4) the improvement of the induced magnetic resonance signals in the second loop receiver coil and the third loop receiver coil on the output signal-to-noise ratio.

A further embodiment of the present invention includes the further steps of:
(1) providing the dual-loop receiver coil unit, with the front end of the second loop receiver coil connected to the back end of the third loop receiver coil and the back end of the second loop receiver coil connected to the front end of the third loop receiver coil, and transferring the received signal of the dual-loop receiver coil unit to a first radiofrequency receiving channel of the system,
(2) combining the detected signals of the first loop receiver coil and the saddle receiver coil into an orthogonal signal firstly by 90° phase shifting, then transferring the orthogonal signal to a second radiofrequency receiving channel of the system, to be processed,
(3) combining the signals from the first and second radiofrequency receiving channels.

In a further embodiment of the present invention, when providing the dual-loop receiver coil unit in the above-described method of the present invention, a second loop receiver coil and a third loop receiver coil independent of each other are employed. The detected signals of the first loop receiver coil and the saddle receiver coil are firstly combined into an orthogonal signal by 90° phase shifting, then the orthogonal signal is transferred into a first radiofrequency receiving channel of the system, to be processed. The received signals of the second loop receiver coil and the third loop receiver coil are transferred respectively into a second radiofrequency receiving channel and a third radiofrequency receiving channel to be processed; and the above three signals then are combined.

In a further embodiment of the method of the present invention, the second loop receiver coil and the third loop receiver coil of the dual-loop receiver coil unit are connected end to end, and the received signals are transferred into a first radiofrequency receiving channel. The detected signals of the first loop receiver coil and the saddle receiver coil are transferred respectively into second and third radiofrequency receiving channels to be processed, and the received signals from the above three radiofrequency receiving channels are combined.

In another embodiment of the method of the present invention, the second loop receiver coil and the third loop receiver coil of the dual-loop receiver coil unit are independent of each other, and the received signals of the two loop receiver coils are transferred respectively into first and second radiofrequency receiving channels to be processed. The detected signals of the first loop receiver coil and the saddle receiver coil are transferred respectively into third and fourth radiofrequency receiving channels to be processed, and the above four received signals are combined.

In the above, the modifiers first, second, third and fourth are used in the proper numerical sequence in describing each embodiment, and do not necessarily correlate from embodiment-to-embodiment.

The above object also is achieved in accordance with the present invention by a signal receiving device for a magnetic resonance imaging system, wherein the direction of the static magnetic field of the magnetic resonance imaging system is along the Z axis direction, including:
(1) a first loop receiver coil surrounding the imaging (detecting) area, for detecting a signal having a selected signal intensity in the middle of the imaging area, wherein the axial direction of the first loop receiver coil is along the Y axis direction;
(2) a saddle receiver coil overlapped on the outside of the first loop receiver coil in the radial direction, for obtaining a signal having a signal intensity of the whole imaging area, wherein the direction of the two opposite surfaces of the saddle receiver coil is along the X axis direction;
(3) a dual-loop receiver coil unit formed by a second loop receiver coil and a third loop receiver coil, wherein the axial direction of the two loop receiver coils is along the Y axis direction and they are respectively disposed on opposite sides along the Y axis direction relative to the first loop receiver coil, and are substantially equally spaced from the first loop receiver coil, for causing the magnetic resonance signal generated by the detected living organism to induce current in the two loop receiver coils of the dual-loop receiver coil unit following the excitation of the signal; the positions of the two coils can be finely adjusted relative to the loop receiver coil, so as to optimize the reception of the magnetic resonance signals;
(4) at least two radiofrequency receiving channels for receiving the respective received signals of the receiver coils;
(5) at least one signal combining device for combining the received signals from the radiofrequency receiving channels.

In a further embodiment of the present invention, the receiving device further includes:
(1) the front end of the second loop receiver coil being connected to the back end of the third loop receiver coil, the back end of the second loop receiver coil being connected to the front end of the third loop receiver coil, and the received signals of the dual-loop receiver coil unit being transferred into a first radiofrequency receiving channel of the system,
(2) a first signal combining device for combining the detected signals of the middle loop receiver coil and the saddle receiver coil into an orthogonal signal by 90° phase shifting,
(3) a first radiofrequency receiving channel for receiving the orthogonal signal from the first signal combiner,
(4) a second radiofrequency receiving channel for receiving the received signals of the dual-loop receiver coil, and
(5) a second signal combining device for combining the signals of the first radiofrequency receiving channel and the second radiofrequency receiving channel.

In another embodiment of the present invention, the receiving device further includes:
(1) the dual-loop receiver coil unit including a second loop receiver coil and a third loop receiver coil, which are independent of each other,
(2) a first signal combining device for combining the detected signals of the first loop receiver coil and the saddle receiver coil into an orthogonal signal by 90° phase shifting,
(3) a first radiofrequency receiving channel for receiving the orthogonal signal from the first signal combiner,
(4) a second radiofrequency receiving channel and a third radiofrequency receiving channel for receiving the received signals of the second loop receiver coil and the third loop receiver coil respectively,
(5) a second signal combining device for combining the signals from the first and second radiofrequency receiving channels.

In another embodiment of the present invention, the receiving device further includes:
(1) the front end of the second loop receiver coil being connected to the back end of the third loop receiver coil, the back end of the second loop receiver coil being connected to the front end of the third loop receiver coil,
(2) the first radiofrequency receiving channel receiving the received signal of said first loop receiver coil,
(3) the second radiofrequency receiving channel receiving the received signal of the saddle receiver coil,
(4) the third radiofrequency receiving channel receiving the received signals of the second loop receiver coil and third loop receiver coil, and
(5) the signal combining device combining the signals of the three radiofrequency receiving channels.

In another embodiment of the present invention, the receiving device further includes:
(1) the counter-rotating loop unit being formed by two loop receiver coils, which are connected end-to-end in series,
(2) a first radiofrequency receiving channel receiving the detected signal of the middle loop receiver coil,
(3) a second radiofrequency receiving channel receiving the detected signal of the saddle receiver coil,
(4) a third radiofrequency receiving channel receiving the detected signal of the counter-rotating loop, and
(5) the signal combining device combining the signals of the three radiofrequency receiving channels.

In another embodiment of the present invention, the receiving device further includes:
(1) the dual-loop receiver coil unit being formed by a second loop receiver coil, and a third loop receiver coil, which are independent of each other,
(2) a first radiofrequency receiving channel receiving the received signal of the first loop receiver coil,
(3) a second radiofrequency receiving channel receiving the received signal of the saddle receiver coil,
(4) a third radiofrequency receiving channel and a fourth radiofrequency receiving channel receiving the received signals of the second loop receiver coil and the third loop receiver coil respectively, and
(5) the signal combining device combining the signals of the above four radiofrequency receiving channels.

### DESCRIPTION OF THE DRAWINGS

FIG. 1 schematically illustrates a first embodiment of an antenna array according to the invention wherein the two loop receiver coils of the counter-rotating loops are connected end-to-end in series.

FIG. 2 schematically illustrates a second embodiment of an antenna array according to the invention wherein the two loop receiver coils of the counter-rotating loops are connected end-to-end in series.

FIG. 3 schematically illustrates a third embodiment of an antenna array according to the invention wherein the two loops receiver coil of the counter-rotating loops are independent from each other.

FIG. 4 schematically illustrates a fourth embodiment of an antenna array according to the invention wherein the two loops receiver coil of the counter-rotating loops are independent from each other.

FIG. 5 shows the signal-to-noise ratio property of the antenna array of the present invention without considering the coupling between the counter-rotating loop receiver coil unit and the first loop receiver coil.

### DESCRIPTION OF THE PREFERRED EMBODIMENTS

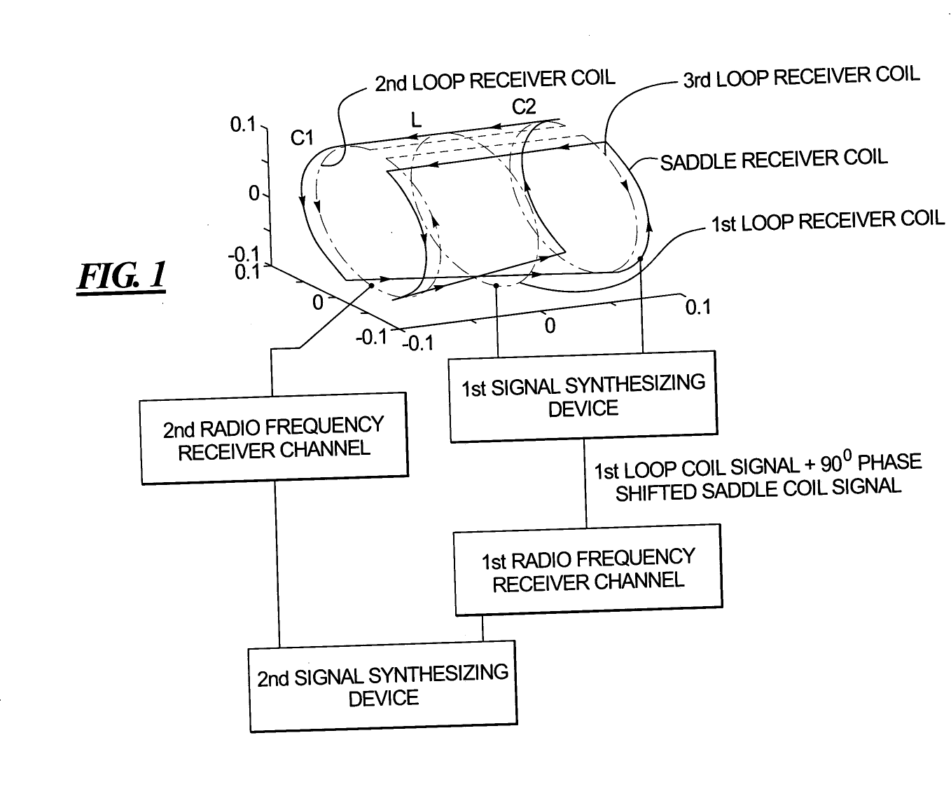

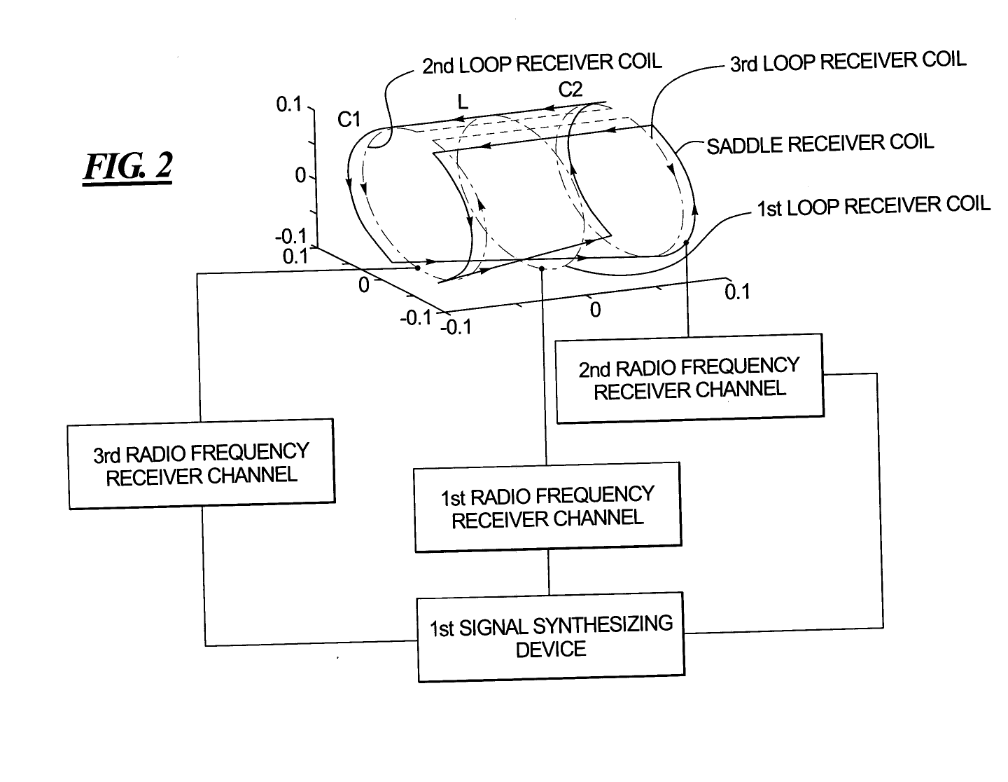

FIGS. 1 and 2 schematically illustrate embodiments of an antenna array of the present invention, respectively, In the Figures, L is a receiver coil set formed by a first loop receiver coil plus a saddle coil; C1 and C2 are two loop receiver coils of a dual-loop receiver coil unit, respectively. The two loop receiver coils C1 and C2 of the dual-loop receiver coil are connected end-to-end in series, i,e. the coil front end of C1 is connected to the coil back end of C2, and the coil back end of C1 is connected to the coil front end of C2. Since the counter-rotating loop is formed by two parallel coil loops symmetrically located on both sides of the original first loop receiver coil, and is located inside the saddle surfaces formed by the saddle coil as the first loop receiver coil, the coil imaging area can be expanded without significantly increasing the volume of the antenna. At the same time, since the counter-rotating loop unit has better signal receiving ability compared to the saddle receiver coil, the imaging quality of the device is improved, especially the imaging quality in the edge portions of the imaging area.

The electrical and structural symmetrical properties of the inventively designed counter-rotating loop unit eliminate the problem of strong coupling between the parallel loop receiver coils, and reduce the coupling between the counter-rotating loops and the first loop receiver coil to a very low level. At the same time, the inventive structure of the counter-rotating loop unit can increase the size of the field of view of the device and enhance the signal-to-noise ratio of the edge portions without changing the original coil volume.

Those skilled in the art can select the winding number of the coils C1, C2, their current spatial relationship between the coils C1, C2 and L, and the spatial relationship between C1 and C2 based on the specific system requirements and associated technical parameters.

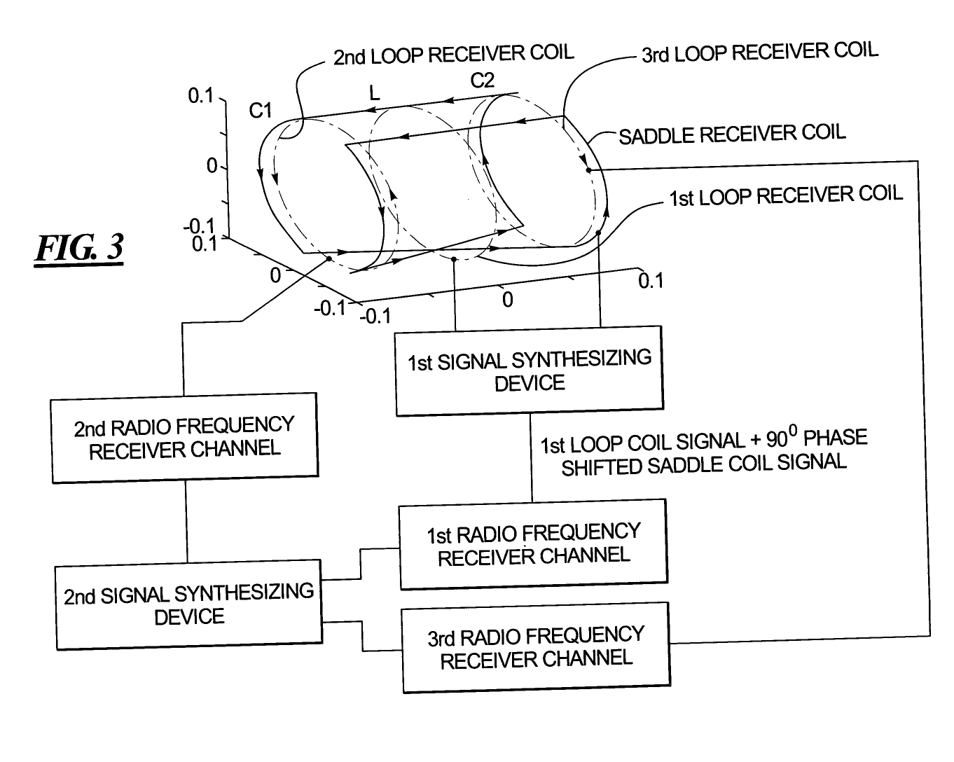

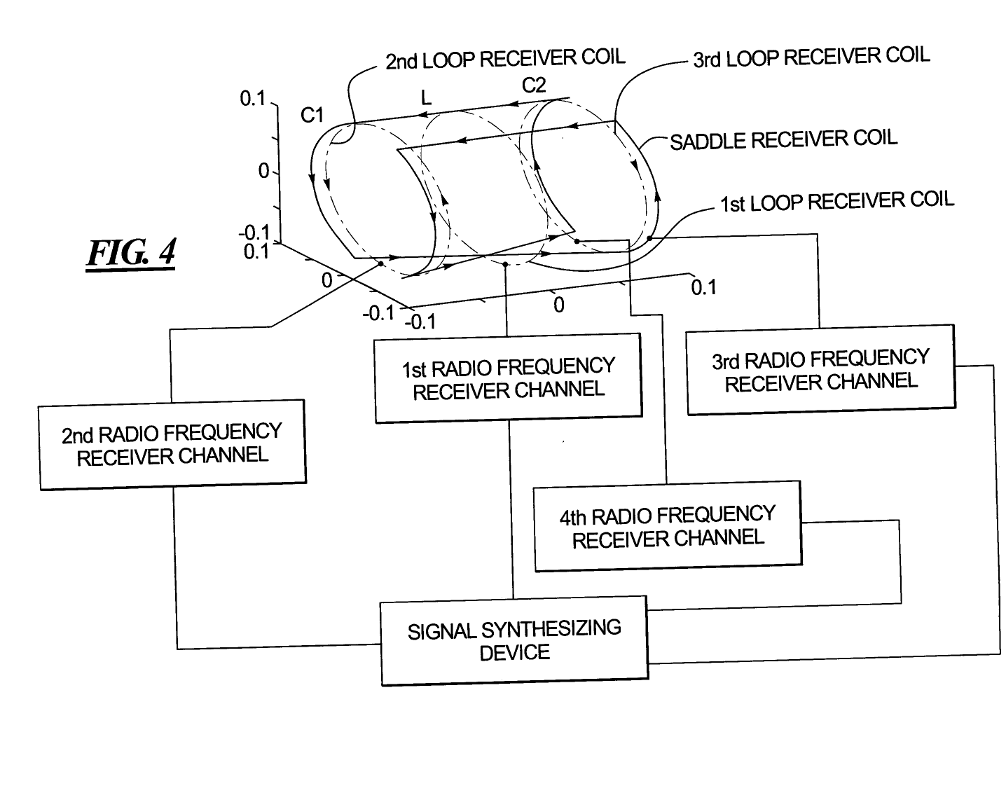

FIGS. 3 and 4 schematically illustrate further embodiments of the antenna array of the present invention respectively. In these figures, the two loop receiver coils C1 and C2 of the dual-loop receiver coil are relatively independent from each other.

In the embodiments shown in FIG. 1 to 4, the output signals of the coil set consisting of the first loop receiver coil and the saddle receiver coil, which coil set is represented by L, can be output via a single radiofrequency receiving channel, or alternatively via two radiofrequency receiving channels, respectively. When the two loop receiver coils C1 and C2 are connected end-to-end, their output signals are emitted via a single radiofrequency receiving channel; when the two coils are independent from each other, their output signals are respectively emitted via two radiofrequency receiving channels. The output signals of L and C1 and C2 emitted via at least one radiofrequency receiving channel, i.e. the output signals of the radiofrequency receiving channel or channels can be combined in a single combining unit.

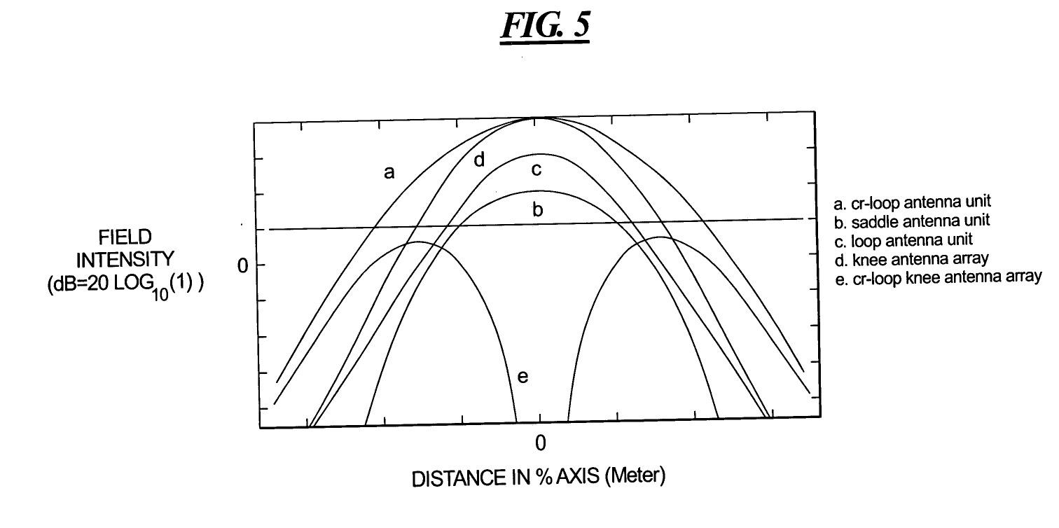

FIG. 5 shows the respective calculated signal-to-noise ratios of the first loop receiver coil, saddle receiver coil and the counter-rotating loop unit of the present invention and their effects on the signal-to-noise ratio index of the whole coil, without considering the coupling between the counter-rotating loop unit and loop receiver coil (worst-case condition). FIG. 5 shows simultaneously the signal-to-noise ratio of the circularly polarized knee antenna array consisting of the first loop receiver coil and the saddle receiver coil, and the signal-to-noise index of the counter-rotating loop knee antenna array formed by the first loop receiver coil, the saddle receiver coil and the counter-rotating loop.

The related physical parameters of coils in FIG. 5 are as follows;

Counter-rotating loop space=16 cm; diameter=10 cm; saddle length=18 cm; saddle opening angle=60°; the crossing line in the chart is the signal attenuation point at 6 dB or 50%.

Referenced to the first loop receiver coil, the signal-to-noise ratio K for the middle portions of the saddle receiver coil and the first loop receiver coil is firstly determined by experiment;
K=SNR_{saddle}(0)/SNRₗₒₒₚ(0)=0.827

Then, the synthesized signal-to-noise ratio for the middle portions of the first loop receiver coil and saddle receiver coil is defined as a reference;

$$\begin{matrix}{SNR}_{ref} = {( {[ {SNR}_{saddle} ( 0 ) ]}^{2} + {[ {SNR}_{loop} ( 0 ) ]}^{2} )}^{1 / 2} \\ = [ K * {SNR}_{saddle} ( 0 ) + {SNR}_{loop} ( 0 ) ] / ( 1 + {K}^{2} ) {,}^{1 / 2}\end{matrix}$$

The respective signal-to-noise ratios of the saddle receiver coil and the counter-rotating loop unit and their effects on the signal-to-noise ratio index of the whole antenna array (FIG. 3) can be calculated without considering the coupling between the counter-rotating loop unit and the first loop receiver coil (worst-case condition).

It can be seen that the imaging area is expanded in Z axis direction without increasing the size of the antenna. If calculated with the signal-to-noise ratio decreased by 6 dB, the imaging area expands a total 5.4 centimeters to both sides, that is, 30% of the antenna length of 18 centimeters; at the same time the signal-to-noise ratio index and the flatness of the signal-to-noise ratio in the imaging area are significantly improved.

The following table briefly shows the advantages of the solution of the present invention over the prior art.

| | Prior art knee | Counter-rotating knee |
|---|---|---|
| Categories | antenna array | antenna array |
| Imaging area | small | Large |
| Signal-to-noise ratio | small | Large |
| Signal homogeneity in | poor | Good |
| imaging area | | |
| Volume | No change | No change |
| Channel number | 1 or 2 | 2 to 4 |

The additional imaging area achieved with the present invention can reach 30% of the antenna volume without increasing the antenna volume, while the coil imaging quality is greatly improved, thus facilitating the positioning of the patients and improving the viewing effect for the pathologically changed portions.

Embodiments of the present invention address the problem of coupling between the counter-rotating loop and the first loop receiver coil by utilizing the symmetry of the counter-rotating loop unit, thereby simplifying the circuit and decreasing the difficulty of adjustment, while optimizing the appearance design of the coil structure.

Therefore, in an embodiment of the present invention, the received signals of the first loop receiver coil and the saddle receiver coil are subjected to 90° phase shifting to be combined into an orthogonal signal, which is input into the radiofrequency receiving channel of the system, to be processed. The received signal of the counter-rotating loop unit is input directly into another radiofrequency receiving channel to be processed; and finally the two signals are combined.

In another embodiment of the present invention, an alternative is to use two independent loop receiver coils (second loop receiver coil and third loop receiver coil) instead of the counter-rotating loop unit for the system with more than two channels. This embodiment can further increase the size of the field of view. This embodiment requires decoupling three loop receiver coils, and the system requirement is relatively high because the system should have at least three radiofrequency receiving channels. For example, if the system has four radiofrequency receiving channels, the received signals of the first, second and third loop receiver coils and the saddle receiver coil can be output and processed respectively via these four radiofrequency receiving channels. Those skilled in the art can easily realize this kind of multi-channel system in accordance with the above description.

Using the method of the present invention, one or more of the following factors should be considered when adjusting the second loop receiver coil and the third loop receiver coil relative to the position of the knee antenna coil to improve the performance of the system;
1) the signal homogeneity of the imaging area of the induced magnetic resonance signal in the second loop receiver coil and the third loop receiver coil,
2) the expansion of the imaging area,
3) the improvement of the imaging of the edge portions of the imaging area,
4) the improvement of the induced magnetic resonance signals in the second loop receiver coil and the third loop receiver coil on the output signal-to-noise ratio.

Based on the above explanations, those skilled in the art will understand how to consider one or more of the above factors to optimize the receiving and detecting effect of the magnetic resonance imaging signals for human or animal organisms subjects

Although modifications and changes may be suggested by those skilled in the art, it is the invention of the inventor to embody within the patent warranted hereon all changes and modifications as reasonably and properly come within the scope of their contribution to the art.

1. A signal receiving method for a magnetic resonance imaging system having an imaging area, wherein a direction of a static magnetic field of the magnetic resonance imaging system is along the Z axis direction, comprising:
providing a first loop receiver coil, surrounding a middle of the imaging area, for detecting the signal intensity in the middle of the imaging area, wherein an axial direction of the first loop receiver coil is along the Y axis direction;
overlapping a saddle receiver coil on the outside of the first loop receiver coil in the radial direction, for obtaining a selected signal intensity of an entirety of the imaging area, wherein a direction of two opposite curved surfaces of the saddle receiver coil is along the X axis direction;
providing a dual-loop receiver coil unit, formed by a second loop receiver coil and a third loop receiver coil, wherein an axial direction of the second and third loop receiver coils is along the Y axis direction and said second and third loop receiver coils are respectively disposed on opposite sides along the Y axis direction relative to the first loop receiver coil, and are substantially equally spaced from the first loop receiver coil, for causing a magnetic resonance signal generated by a subject in the imaging area to induce current in the second and third loop receiver coils of the dual-loop receiver coil unit following excitation of the magnetic resonance signal;
supplying respectively detected signals of the first loop receiver coil, the saddle receiver coil, and the second and third loop receiver coil units to at least one radiofrequency receiving channel and combining al of the detected signals; and
adjusting positions of the second loop receiver coil and the third loop receiver coil relative to the first loop receiver coil for optimizing reception of the magnetic resonance signal of the detected living organism, dependent on at least one of the following factors:
signal homogeneity of the imaging area of the induced magnetic resonance signals in the second loop receiver coil and the third loop receiver coil,
expansion of the imaging area,
improvement of imaging of edge portions of the imaging area, and
improvement of the induced magnetic resonance signals in the second loop receiver coil and the third loop receiver coil on an output signal-to-noise ratio thereof.

2. A method as claimed in claim 1 comprising:
providing the dual-loop receiver coil unit with a front end of the second loop receiver coil connected to a back end of the third loop receiver coil and a back end of the second loop receiver coil connected to a front end of the third loop receiver coil, and transferring the received signal of the dual-loop receiver coil unit to a first radiofrequency receiving channel of the system;
combining the detected signals of the first loop receiver coil and the saddle receiver coil into an orthogonal signal by 90° phase shifting, then transferring the orthogonal signal to a second radiofrequency receiving channel of the system; and
combining the signals from the first and second radiofrequency receiving channels.

3. A method as claimed in claim 1 comprising providing the dual-loop receiver coil unit with the second loop receiver coil and a third loop receiver coil independent of each other, combining the detected signals of the first loop receiver coil and the saddle receiver coil into an orthogonal signal by 90° phase shifting, then transferring the orthogonal signal into a first radiofrequency receiving channel of the system, transferring the received signals of the second loop receiver coil and the third loop receiver coil respectively into a second radiofrequency receiving channel and a third radiofrequency receiving channel; and combining the respective signals from the first, second and third radiofrequency channels.

4. A method as claimed in claim 1 comprising providing the dual-loop receiver coil unit with the second loop receiver coil and the third loop receiver coil connected end to end, and transferring the received signals into a first radiofrequency receiving channel of the system, transferring the detected signals of the first loop receiver coil and the saddle receiver coil respectively into second and third radiofrequency receiving channels of the system, and combining the received signals from the first, second and third radiofrequency receiving channels.

5. A method as claimed in claim 1 comprising providing the dual-loop receiver coil unit with the second loop receiver coil and the third loop receiver coil independent of each other, transferring the received signals of the second and third loop receiver coils respectively into first and second radiofrequency receiving channels of the system, transferring the detected signals of the first loop receiver coil and the saddle receiver coil respectively into third and fourth radiofrequency receiving channels of the system, and combining the received signals from the first, second, third and fourth radiofrequency receiving channels.

6. A signal receiving device for a magnetic resonance imaging system having an imaging area, wherein a direction of a static magnetic field of the magnetic resonance imaging system is along the Z axis direction, comprising:
a first loop receiver coil surrounding the imaging area, for detecting a signal having a selected signal intensity in a middle of the imaging area, wherein an axial direction of the first loop receiver coil is along the Y axis direction;
a saddle receiver coil overlapped on the outside of the first loop receiver coil in a radial direction, for obtaining a selected signal intensity of an entirety of the imaging area, wherein a direction of two opposite surfaces of the saddle receiver coil is along the X axis direction;
a dual-loop receiver coil unit formed by a second loop receiver coil and a third loop receiver coil, wherein an axial direction of the second and third loop receiver coils is along the Y axis direction and the second and third receiver coils are respectively disposed on opposite sides along the Y axis direction relative to the first loop receiver coil, and are substantially equally spaced from the first loop receiver coil, for causing a magnetic resonance signal generated by a subject in the imaging area to induce current in the second and third loop receiver coils of the dual-loop receiver coil unit following excitation of the magnetic resonance signal; the positions of the second and third loop receiver coils being finely adjusted relative to the loop receiver coil, to optimize reception of the magnetic resonance signals;
at least two radiofrequency receiving channels for receiving the received signals of the first, second and third receiver coils; and
at least one signal combining device for combining the received signals from the radiofrequency receiving channels.

7. A receiving device as claimed in claim 6 comprising:
a front end of the second loop receiver coil being connected to a back end of the third loop receiver coil, a back end of the second loop receiver coil being connected to a front end of the third loop receiver coil, and the received signals of the dual-loop receiver coil unit being transferred into a first radiofrequency receiving channel;
a first signal combining device for combining the detected signals of the middle loop receiver coil and the saddle receiver coil into an orthogonal signal by 90° phase shifting;
a first radiofrequency receiving channel for receiving the orthogonal signal from the first signal combiner;
a second radiofrequency receiving channel for receiving the received signals of the dual-loop receiver coil; and
a second signal combining device for combining the signals of the first radiofrequency receiving channel and the second radiofrequency receiving channel.

8. A receiving device as claimed in claim 6 comprising:
the second loop receiver coil and the third loop receiver coil in the dual-loop receiver coil unit being independent of each other;
a first signal combining device for combining the detected signals of the first loop receiver coil and the saddle receiver coil into an orthogonal signal by 90° phase shifting;
a first radiofrequency receiving channel receiving the orthogonal signal from the first signal combiner;
a second radiofrequency receiving channel and a third radiofrequency receiving channel for receiving the received signals of the second loop receiver coil and the third loop receiver coil respectively; and
a second signal combining device for combining the signals from the first and second radiofrequency receiving channels.

9. A receiving device as claimed in claim 6 comprising:
a front end of the second loop receiver coil being connected to a back end of the third loop receiver coil, a back end of the second loop receiver coil being connected to a front end of the third loop receiver coil;
a first radiofrequency receiving channel receiving the received signal of said first loop receiver coil;
a second radiofrequency receiving channel receiving the received signal of the saddle receiver coil;
a third radiofrequency receiving channel receiving the received signals of the second loop receiver coil and third loop receiver coil, and the signal combining device combining the signals of the first, second and third radiofrequency receiving channels.

10. A receiving device as claimed in claim 6 comprising:
a counter-rotating loop unit formed by two loop receiver coils, which are connected end-to-end in series;
a first radiofrequency receiving channel receiving the detected signal of the middle loop receiver coil;
a second radiofrequency receiving channel receiving the detected signal of the saddle receiver coil;
a third radiofrequency receiving channel receiving the detected signal of the counter-rotating loop; and
the signal combining device combining the signals of the first, second and third radiofrequency receiving channels.

11. A receiving device as claimed in claim 6 comprising:
a dual-loop receiver coil unit formed by a second loop receiver coil, and a third loop receiver coil, which are independent of each other;
a first radiofrequency receiving channel receiving the received signal of the first loop receiver coil;
a second radiofrequency receiving channel receiving the received signal of the saddle receiver coil;
a third radiofrequency receiving channels and a fourth radiofrequency receiving channel receiving the received signals of the second loop receiver coil and the third loop receiver coil respectively; and
the signal combining device combining the signals of the first, second, third and fourth radiofrequency receiving channels.
